(19) United States
(12) Patent Application Publication
(10) Pub. No.: US 2025/0170029 A1
(43) Pub. Date: May 29, 2025

(54) HYDROCOLLOID SHEET WITH EXFOLIATION PERFORMANCE

(71) Applicant: SKIN SOLUTION CO., LTD, Ansan-si (KR)

(72) Inventor: Yoo Sik NA, Suwon-si (KR)

(21) Appl. No.: 18/756,063

(22) Filed: Jun. 27, 2024

(30) Foreign Application Priority Data
Nov. 24, 2023 (KR) 10-2023-0166100

(51) Int. Cl. A61K 8/02; A61K 8/368; A61K 8/38; A61Q 19/10; B32B 7/06; B32B 27/08; B32B 27/36; B32B 27/40; B32B 37/24

(52) U.S. Cl. CPC A61K 8/0208; A61K 8/368; A61K 8/38; A61Q 19/10; B32B 7/06; B32B 27/08; B32B 27/36; B32B 27/40; B32B 37/24; B32B 2037/243; B32B 2250/02; B32B 2250/24; B32B 2255/10; B32B 2255/24; B32B 2255/26; B32B 2255/28; B32B 2307/726; B32B 2307/7376; B32B 2307/748; B32B 2555/00

(57) ABSTRACT

Proposed is a hydrocolloid sheet with exfoliation performance, the hydrocolloid sheet including a release layer, a hydrocolloid layer stacked on an upper surface of the release layer and including a pressure-sensitive adhesive, an absorbent, and an exfoliant, and a base layer staked on an upper surface of the hydrocolloid layer and protecting the hydrocolloid layer.

### CROSS REFERENCE TO RELATED APPLICATION

The present application claims priority to Korean Patent Application No. 10-2023-0166100, filed Nov. 24, 2023, the entire contents of which is incorporated herein for all purposes by this reference.

### BACKGROUND

### Field of the Invention

The present disclosure relates to a hydrocolloid sheet with exfoliation performance.

### Description of the Related Art

Dead skin cells cover the skin of the body and serve as a barrier that prevents evaporation of moisture from the skin and entry of external foreign substances into the body. However, when excessive dead skin cells accumulate on the skin surface, they cause rough skin texture and dull skin tone. In addition, they impede absorption of skincare nutrients into the skin.

When dead skin cells and keratin plugs are left on the skin surface, they clog pores, leading to various problems such as acne disease, and dead cells and irregular melanin pigmentation in the stratum corneum of the skin cause aesthetic problems. These dead skin cells and keratin plugs cannot be removed even when the face is washed thoroughly, and even commonly used facial cleansers are not sufficient to completely remove the dead skin cells deep within pores. In addition, when the skin does not retain a certain level of moisture and becomes dry, it can lead to itching, and in severe cases, is prone to forming fine cracks. In this case, harmful bacteria can enter the skin through the cracks and cause skin diseases, so it is necessary to manage dead skin cells.

Conventional methods for removing dead skin cells or keratin plugs from the skin surface include a method of removing keratin plugs using cationic polymers and a method of removing keratin plugs using a sheet-type cosmetic composition composed of a film-forming cosmetic layer and a moisture-permeable substrate. However, despite of their effectiveness in removing dead skin cells and keratin plugs, these conventional methods are not only inconvenient to implement or use, but also problematic in that they cannot be carried around and remove unnecessary dead skin cells or keratin plugs from the skin surface at the desired time. Therefore, there is a need to develop a pressure-sensitive adhesive sheet for removing dead skin cells and keratin plugs that can effectively and easily remove unnecessary dead skin cells and keratin plugs from the skin surface without causing a burden on the skin.

The foregoing is intended merely to aid in the understanding of the background of the present disclosure, and is not intended to mean that the present disclosure falls within the purview of the related art that is already known to those skilled in the art.

### DOCUMENTS OF RELATED ART

(Patent Document 1) Korean Patent Application Publication No. 10-2011-0113904 A (Publication Date: Oct. 19, 2011)

### SUMMARY

Accordingly, the present disclosure has been made keeping in mind the above problems occurring in the related art, and an objective of the present disclosure is to provide a hydrocolloid sheet with exfoliation performance that can remove unnecessary dead skin cells and keratin plugs from the skin surface by simply attaching the sheet to the skin.

In order to achieve the above objective, according to one aspect of the present disclosure, there is provided a hydrocolloid sheet with exfoliation performance, the hydrocolloid sheet including: a release layer; a hydrocolloid layer stacked on an upper surface of the release layer and including a pressure-sensitive adhesive, an absorbent, and an exfoliant; and a base layer staked on an upper surface of the hydrocolloid layer and protecting the hydrocolloid layer. In addition, the hydrocolloid layer may include 20 to 40 parts by weight of the absorbent and 0.1 to 15 parts by weight of the exfoliant, with respect to 100 parts by weight of the pressure-sensitive adhesive.

In addition, the pressure-sensitive adhesive may be one or more selected from the group consisting of a styrene isoprene styrene block copolymer (SIS), a styrene isoprene styrene block copolymer (SI), a styrene ethylene butylene styrene block copolymer (SEBS), a styrene butadiene styrene block copolymer (SBS), and polyisobutylene (PIB).

In addition, the absorbent may be one or more selected from the group consisting of sodium carboxymethyl cellulose, cellulose gum, carbomer, xantham gum, gelatin, collagen, pectin, hyaluronic acid, guar gum, sodium alginate, agar, konjac, and arabic gum.

In addition, the exfoliant may be one or more selected from the group consisting of salicylic acid, benzoyl peroxide, glycolic acid, lactic acid, malic acid, citric acid, and tartaric acid.

In addition, the exfoliant may be salicylic acid or benzoyl peroxide.

In addition, the release layer may have a thickness of 50 to 150 μm.

In addition, the hydrocolloid layer may have a thickness of 100 to 600 μm.

In addition, the base layer may have a thickness of 10 to 50 μm.

In addition, the base layer may be one or more selected from the group consisting of polyolefin (PO), polyvinyl alcohol (PVA), polystyrene (PS), polyvinyl chloride (PVC), polyurethane (PU), and polyethylene terephthalate (PET).

According to another aspect of the present disclosure, there is provided a method of manufacturing a hydrocolloid sheet with exfoliation performance, the method including: (a) preparing a mixture by mixing 20 to 40 parts by weight of an absorbent and 0.1 to 15 parts by weight of an exfoliant with 100 parts by weight of a pressure-sensitive adhesive; (b) coating the mixture of step (a) on the release layer to form a hydrocolloid layer; and (c) forming a base layer on an upper surface of the hydrocolloid layer of step (b).

According to the present disclosure, it is possible to provide a hydrocolloid sheet with exfoliation performance that can remove unnecessary dead skin cells and keratin plugs from the skin surface by simply attaching the sheet to the skin.

In addition, it is possible to provide a hydrocolloid sheet with exfoliation performance that has excellent adhesion to the skin.

In addition, it is possible to provide a hydrocolloid sheet with exfoliation performance that does not cause irritation to the skin.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a view illustrating a hydrocolloid sheet with exfoliation performance according to an embodiment of the present disclosure;

FIG. 2 is a flowchart illustrating a method of manufacturing a hydrocolloid sheet with exfoliation performance according to another embodiment of the present disclosure;

FIG. 3 is a view illustrating the results of analyzing the dead skin cell improvement effect of sheets manufactured according to Examples and Comparative Examples; and

FIG. 4 is a view illustrating the results of analyzing the water absorption of sheets manufactured according to Examples and Comparative Examples.

### DETAILED DESCRIPTION

In one embodiment, the present disclosure provides a hydrocolloid sheet with exfoliation performance, the hydrocolloid sheet including a release layer, a hydrocolloid layer stacked on an upper surface of the release layer and including a pressure-sensitive adhesive, an absorbent, and an exfoliant, and a base layer staked on an upper surface of the hydrocolloid layer and protecting the hydrocolloid layer.

Hereinbelow, exemplary embodiments of the present disclosure will be described in detail with reference to the accompanying drawings such that the present disclosure can be easily embodied by one of ordinary skill in the art to which the present disclosure belongs. Various changes to the following embodiments are possible and the scope of the present disclosure is not limited to the following embodiments. Wherever possible, the same reference numerals will be used throughout the drawings and the description to refer to the same or like elements or parts.

In one embodiment, the present disclosure provides a hydrocolloid sheet with exfoliation performance, the hydrocolloid sheet including a release layer 100, a hydrocolloid layer 200 stacked on an upper surface of the release layer 100 and including a pressure-sensitive adhesive, an absorbent, and an exfoliant, and a base layer 300 staked on an upper surface of the hydrocolloid layer 200 and protecting the hydrocolloid layer 200.

The release layer 100 is a support that supports the hydrocolloid layer 200, and at the serves to prevent entry of external contaminants and protects the hydrocolloid layer 200 from external shock.

The release layer 100 may be one or more selected from the group consisting of polyolefin (PO), polyvinyl alcohol (PVA), polystyrene (PS), polyvinyl chloride (PVC), polyurethane (PU), and polyethylene terephthalate (PET), and may specifically be PET.

The release layer 100 is in the form of a film, and may specifically be a film made of PET, and more specifically, may be a silicone release-treated PET film.

The manufacturing method of the release layer 100 is not limited, and commercially available PET film may be purchased and used.

In addition, the thickness of the release layer 100 is not particularly limited, but the average thickness of the release layer 100 may be 50 to 150 μm, specifically 80 to 120 μm, and more specifically 90 to 110 μm.

When the average thickness of the release layer 100 is less than 50 μm, its function of protecting the hydrocolloid layer 200 from external shock may be reduced, it has a poor appearance, and workability is reduced when forming the hydrocolloid layer 200 on the release layer 100. When the average thickness of the release layer 100 exceeds 150 μm, flexibility is reduced.

The hydrocolloid layer 200 may be formed by coating on the upper surface of the release layer 100. In addition, the hydrocolloid layer 200 may include a pressure-sensitive adhesive, an absorbent, and an exfoliant.

The pressure-sensitive adhesive may be one selected from the group consisting of a styrene isoprene styrene block copolymer (SIS), a styrene isoprene styrene block copolymer (SI), a styrene ethylene butylene styrene block copolymer (SEBS), a styrene butadiene styrene block copolymer (SBS), and polyisobutylene (PIB), and may specifically be SIS.

The absorbent may be one or more selected from the group consisting of sodium carboxymethyl cellulose (CMC), cellulose gum, carbomer, xantham gum, gelatin, collagen, pectin, hyaluronic acid, guar gum, sodium alginate, agar, konjac, and arabic gum, and may specifically be CMC.

The exfoliant may be one or more selected from the group consisting of salicylic acid, benzoyl peroxide, glycolic acid, lactic acid, malic acid, citric acid, and tartaric acid, and may specifically be salicylic acid or benzoyl peroxide.

The hydrocolloid layer 200 may include 20 to 40 parts by weight of the absorbent and 0.1 to 15 parts by weight of the exfoliant, with respect to 100 parts by weight of the pressure-sensitive adhesive.

For example, when salicylic acid is used as the exfoliant, the content of the exfoliant may be 1 to 2 parts by weight, and when benzoyl peroxide is used as the exfoliant, the content of the exfoliant may be 0.1 to 15 parts by weight.

When the content of the exfoliant exceeds the above range, the exfoliation effect may not be obtained or side effects such as skin trouble may occur.

When the content of the absorbent is less than 20, the effectiveness of hydrocolloid may be reduced due to low absorption of wound fluid. When the content of the absorbent exceeds 40 parts by weight, adhesion may be reduced. Therefore, the above range is preferable.

In addition, the hydrocolloid sheet can exhibit a further improved exfoliation effect when it includes the pressure-sensitive adhesive, absorbent, and exfoliant in the above-described range.

The pressure-sensitive adhesive may have a pressure-sensitive adhesive strength of 500 to 2,000 gf/25 mm.

When the pressure-sensitive adhesive strength is less than 500 gf/25 mm, the exfoliation effect and adhesion may decrease due to a reduction in pressure-sensitive adhesive performance. When the pressure-sensitive adhesive strength exceeds 2,000 gf/25 mm, the hydrocolloid sheet may cause skin damage when peeled off from the face.

The average thickness of the hydrocolloid layer 200 may be 100 to 600 μm, specifically 150 to 350 μm, and more specifically 300 μm.

When the average thickness of the hydrocolloid layer 200 is less than 100 μm, the exfoliation effect may be reduced. When the average thickness of the hydrocolloid layer 200 exceeds 600 μm, the overall thickness of the sheet may be increased, productivity may be reduced, and flexibility may be reduced.

The base layer 300 may be formed on the upper surface of the hydrocolloid layer 200 to serve to protect the hydrocolloid layer 200 from external contaminates or external shock and smooth out facial wrinkles.

The base layer 300 may be one or more selected from the group consisting of polyolefin (PO), polyvinyl alcohol (PVA), polystyrene (PS), polyvinyl chloride (PVC), polyurethane (PU), and polyethylene terephthalate (PET), and may specifically be PU.

For example, the hydrocolloid sheet may be a sheet in which the hydrocolloid layer 200 is formed between the release layer 100 made of PET and the base layer 300 made of PU.

The base layer 300 is in the form of a film, and may specifically be a silicone release-treated film.

The manufacturing method of the base layer 300 is not limited.

In addition, the thickness of the base layer 300 is not particularly limited, but the average thickness of the base layer 300 may be 10 to 50 μm, specifically 15 to 30 μm, and more specifically 20 μm.

When the average thickness of the base layer 300 is less than 10 μm, its function of protecting the hydrocolloid layer 200 may be reduced. When the average thickness of the base layer 300 exceeds 50 μm, flexibility may be reduced, resulting in poor wearing comfort.

In another embodiment, the present disclosure provides a method of manufacturing a hydrocolloid sheet with exfoliation performance, the method including (a) preparing a mixture by mixing 20 to 40 parts by weight of an absorbent and 0.1 to 15 parts by weight of an exfoliant with 100 parts by weight of a pressure-sensitive adhesive, (b) coating the mixture of step (a) on the release layer 100 to form a hydrocolloid layer 200, and (c) forming a base layer 300 on an upper surface of the hydrocolloid layer 200 of step (b).

### Step (a) of Preparing the Mixture

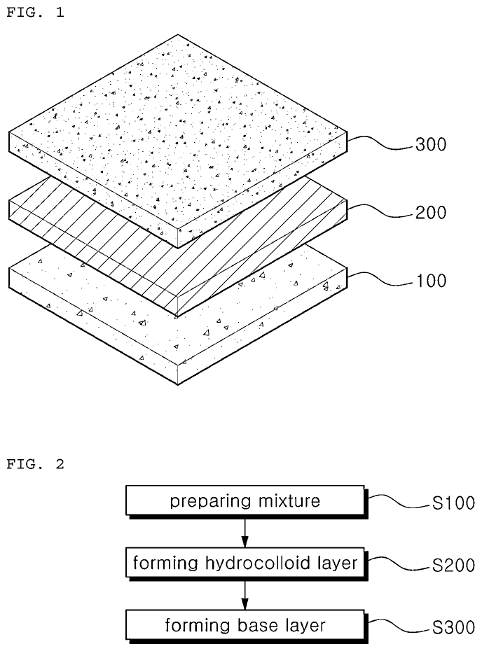

As illustrated in FIG. 2, a step S100 of preparing the mixture is performed.

In step (a), the mixture may be prepared by mixing 20 to 40 parts by weight of the absorbent and 0.1 to 15 parts by weight of the exfoliant with 100 parts by weight of the pressure-sensitive adhesive.

The mixing method is not limited as long as it can sufficiently mix all ingredients.

### Step (b) Forming the Hydrocolloid Layer

Next, as illustrated in FIG. 2, a step S200 of forming the hydrocolloid layer 200 is performed.

The mixture prepared in step (a) may be coated on the release layer 100 to form the hydrocolloid layer 200 on the upper surface of the release layer 100.

Here, the thickness of the release layer 100 is not particularly limited, but a release layer 100 having an average thickness of 50 to 150 μm, specifically 80 to 120 μm, and more specifically 90 to 110 μm may be used.

The release layer 100 may be a silicone release-treated film.

The method of coating the mixture on the release layer 100 is not limited, and the mixture may be coated so that the average thickness of the hydrocolloid layer 200 is 100 to 600 μm, specifically 150 to 350 μm, and more specifically 300 μm.

When the average thickness of the hydrocolloid layer 200 is less than 100 μm, the exfoliation effect may be reduced. When the average thickness of the hydrocolloid layer 200 exceeds 600 μm, the overall thickness of the sheet may be increased, productivity may be reduced, and flexibility may be reduced.

The release layer 100 may be one or more selected from the group consisting of polyolefin (PO), polyvinyl alcohol (PVA), polystyrene (PS), polyvinyl chloride (PVC), polyurethane (PU), and polyethylene terephthalate (PET), and may specifically be PET.

The manufacturing method of the release layer 100 is not limited.

### Step (c) Forming the Base Layer

Finally, as illustrated in FIG. 2, a step S300 of forming the base layer 300 is performed.

In step (c), the base layer 300 may be formed by stacking the base layer 300 in the form of a film on the hydrocolloid layer 200.

The base layer 300 and the hydrocolloid layer 200 may be easily bonded together by the pressure-sensitive adhesive included in the hydrocolloid layer 200.

Here, the base layer 300 may be a silicone release-treated film.

In addition, the thickness of the base layer 300 is not particularly limited, but a base layer 300 having an average thickness of 10 to 50 μm, specifically 15 to 30 μm, and more specifically 20 μm may be used.

Hereinafter, examples of the present disclosure will be described in more detail. These examples are merely for illustrative purposes, and it is obvious to those skilled in the art that the scope of the present disclosure is not limited thereto.

### EXAMPLES

### Example 1

A mixture was prepared by mixing 30 parts by weight of CMC and 2 parts by weight of salicylic acid with 100 parts by weight of SIS, and then the resulting mixture was coated to a thickness of 300 μm on an upper surface of a silicone release-treated PET film with a thickness of 100 μm to form a hydrocolloid layer. Thereafter, a PU film with a thickness of 20 μm was stacked on an upper surface of the hydrocolloid layer to manufacture a hydrocolloid sheet with exfoliation performance.

### Example 2

A hydrocolloid sheet with improved exfoliation performance was prepared in the same manner as in Example 1, except that 10 parts by weight of benzoyl peroxide was used instead of salicylic acid.

### COMPARATIVE EXAMPLES

### Comparative Example 1

A hydrocolloid sheet was prepared in the same manner as in Example 1, except that salicylic acid included in a hydrocolloid layer was not used.

### Comparative Example 2

A hydrocolloid sheet was prepared in the same manner as in Example 1, except that CMC included in a hydrocolloid layer was not used.

### TEST EXAMPLES

### Test Example 1: Analysis of Dead Skin Cell Improvement Effect

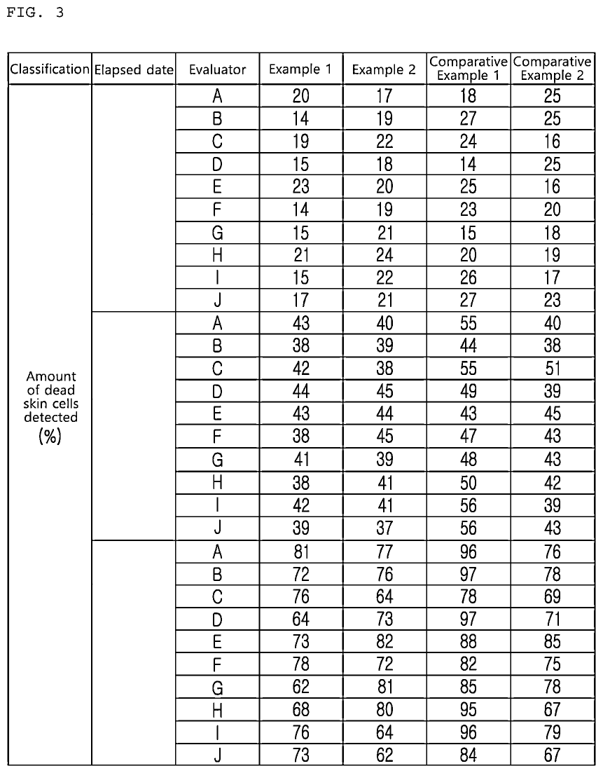

The dead skin cell improvement effect of the hydrocolloid sheets manufactured according to Examples 1 and 2 and Comparative Examples 1 and 2 was analyzed, and the results are illustrated in FIG. 3.

The test methods therefor are as follows.

10 evaluators aged 25 to 35 were asked to clean a test area (face) with a mild cleanser, wait for 20 minutes after 2 hours under measurement environmental conditions of a temperature of 22° C. and a relative humidity of 50%, and expose the test area 10 minutes before measurement. Dead skin cells from the maxillary cheek area of the evaluators were collected using keratin tape.

Samples of Examples and Comparative Examples were simultaneously attached to a location close to a keratin tape collection area and removed after 24 hours. Dead skin cells were collected from the corresponding location using keratin tape. After 3 and 7 days of evaluation, the evaluators washed his/her face with a mild cleanser and dead skin cells were collected from the same evaluation area again. The area of dead skin cells removed from the keratin tape was analyzed using Image-Pro Analyzer, and the dead skin cell improvement effect was expressed as the amount of dead skin cells detected (%).

Referring to FIG. 3, as a result of analyzing the amount of dead skin cells detected, it can be seen that the hydrocolloid sheets manufactured according to Examples 1 and 2 have a superior effect than those of Comparative Examples 1 and 2.

### Test Example 2: Evaluation of Absorption

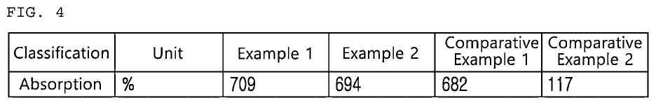

The absorption of the hydrocolloid sheets manufactured according to Examples 1 and 2 and Comparative Examples 1 and 2 was analyzed, and the results are illustrated in FIG. 4.

The absorption evaluation methods therefor are as follows.

Each sheet was cut to a size of 2 cm in width and length, weighed to measure an initial weight (W1), placed in distilled water at 37° C., and weighed to measure a weight (W2) after 48 hours. The absorption (%) was calculated according to Equation 1 below.

### Equation 1

Absorption=(W2/W1)×100

Referring to FIG. 4, it can be seen that the absorption of Comparative Example 1 excluding salicylic acid is lower than that of Examples 1 and 2, and in particular, the absorption of Comparative Example 2 excluding CMC is drastically lowered.

In other words, this indicates that the absorption is further improved when salicylic acid and CMC are used together.

According to the present disclosure, there is provided a hydrocolloid sheet with exfoliation performance that can remove unnecessary dead skin cells and keratin plugs from the skin surface by simply attaching the sheet to the skin.

While a particular embodiment of the present hydrocolloid sheet with exfoliation performance has been described herein, it will be appreciated by those skilled in the art that changes and modifications may be made thereto without departing from the invention in its broader aspects and as set forth in the following claims.

What is claimed is:

1. A hydrocolloid sheet with exfoliation performance, the hydrocolloid sheet comprising:
a release layer;
a hydrocolloid layer stacked on an upper surface of the release layer and including a pressure-sensitive adhesive, an absorbent, and an exfoliant; and
a base layer stacked on an upper surface of the hydrocolloid layer and protecting the hydrocolloid layer.

2. The hydrocolloid sheet of claim 1, wherein the hydrocolloid layer includes 20 to 40 parts by weight of the absorbent and 0.1 to 15 parts by weight of the exfoliant, with respect to 100 parts by weight of the pressure-sensitive adhesive.

3. The hydrocolloid sheet of claim 1, wherein the pressure-sensitive adhesive is one or more selected from the group consisting of a styrene isoprene styrene block copolymer (SIS), a styrene isoprene styrene block copolymer styrene ethylene butylene styrene block copolymer (SEBS), a styrene butadiene styrene block copolymer (SBS), and polyisobutylene (PIB).

4. The hydrocolloid sheet of claim 1, wherein the absorbent is one or more selected from the group consisting of sodium carboxymethyl cellulose, cellulose gum, carbomer, xantham gum, gelatin, collagen, pectin, hyaluronic acid, guar gum, sodium alginate, agar, konjac, and arabic gum.

5. The hydrocolloid sheet of claim 1, wherein the exfoliant is one or more selected from the group consisting of salicylic acid, benzoyl peroxide, glycolic acid, lactic acid, malic acid, citric acid, and tartaric acid.

6. The hydrocolloid sheet of claim 1, wherein the exfoliant is salicylic acid or benzoyl peroxide.

7. The hydrocolloid sheet of claim 1, wherein the release layer has a thickness of 50 to 150 μm.

8. The hydrocolloid sheet of claim 1, wherein the hydrocolloid layer has a thickness of 100 to 600 μm.

9. The hydrocolloid sheet of claim 1, wherein the base layer has a thickness of 10 to 50 μm.

10. The hydrocolloid sheet of claim 1, wherein the base layer is one or more selected from the group consisting of polyolefin (PO), polyvinyl alcohol (PVA), polystyrene (PS), polyvinyl chloride (PVC), polyurethane (PU), and polyethylene terephthalate (PET).

11. A method of manufacturing a hydrocolloid sheet with exfoliation performance, the method comprising:
(a) preparing a mixture by mixing 20 to 40 parts by weight of an absorbent and 0.1 to 15 parts by weight of an exfoliant with 100 parts by weight of a pressure-sensitive adhesive;
(b) coating the mixture of step (a) on the release layer to form a hydrocolloid layer; and
(c) forming a base layer on an upper surface of the hydrocolloid layer of step (b).
